(19) United States
(12) Patent Application Publication
(10) Pub. No.: US 2002/0077679 A1
(43) Pub. Date: Jun. 20, 2002

(54) Device having far infrared radiator for enhancing growth of hair

(76) Inventor: Hao Wen Lo, Tao Yuan City (TW)

Correspondence Address: Rosenberg, Klein & Lee, Suite 105, 3444 Ellicott Center Drive, Ellicot City, MD, 21043, US

(21) Appl. No.: 09/737,577

(22) Filed: Dec. 18, 2000

(51) Int. Cl. A61N 5/06

(52) U.S. Cl. 607/90

(57) ABSTRACT

The present invention proposes a device having a far IR radiator for enhancing the growth of hair, which device comprises a main body having a hold part and a contact part. A battery is installed in the hold part. A far IR radiator is installed in the contact part. A transparent cover is further arranged on the outer surface of the contact part. A comb having arrays of comb fingers is joined on the cover. The main body has a switch and a power socket. The battery will let the far IR radiator emit far IR lights to stimulate the skin of the scalp. Therefore, the hairs can be protected and nourished, and hair follicles can be activated to grow new hairs so that the hairs will be blacker and denser and have more vitality.

### FIELD OF THE INVENTION

The present invention relates to a device having a far infrared (IR) radiator for enhancing the growth of hair and, more particularly, to a far IR structure attached on a comb.

### BACKGROUND OF THE INVENTION

Nowadays, people suffer more and more pressures from work, learning, or even lives. Additionally, in modern computer age, physical labor decreases gradually, while headwork increases unlimitedly. Therefore, more and more people have the problem of calvities. In addition to genetic factors, calvities occur earlier because of pressure. Although hair restorers and hair implantation techniques have been available, some are inappropriate to predisposition and some are expensive so that the problem of calvities cannot be effectively resolved. Furthermore, some people have the annoying problem of gray hairs growing among black hairs. There is also the problem of hair treatment. Almost everybody wants to have raven tresses. Therefore, much money is spent for hair treatment. In prior art, combs are used only for combing or winding hairs, and cannot be used to change the physiological state below the scalp. However, according to recent researches, pressure is one factor resulting in variation of hair quality. The present invention aims to resolve the above problems in prior art.

### SUMMARY OF THE INVENTION

The primary object of the present invention is to provide a device having a far IR radiator for enhancing the growth of hair. Far IR lights have been used for remedy for a long time, and have been proved to have the effect of enhancing the circulation of human body under proper control. Not contacting the human body, far IR lights still have good effect for positions at 1 centimeter below the skin. Based on this idea, a far IR radiator is attached on a tool suitable to the head in the present invention so as to enhance the hair quality and to replenish the hairs. Thereby, the hypodermic hair follicles of the head will be more vital, the activated skin will be more resilient, and dormant hair follicles will be refreshed to grow new hairs. Original hair follicles will have more vitality to absorb and transport nutriments so that the hairs will be denser, softer, and blacker. Therefore, both the effects of hair treatment and hair implantation can be achieved.

To achieve the above object, a device having a far IR radiator for enhancing the growth of hair of the present invention comprises a main body having a hold part and a contact part. A battery is installed in the hold part. A far IR radiator is installed in the contact part. A transparent cover is further arranged on the outer surface of the contact part. A comb having arrays of comb fingers is joined on the cover. The main body has a switch and a power socket. The battery will let the far IR radiator emit far IR lights to stimulate the skin of the scalp so as to protect and nourish the hairs and to activate hair follicles to grow new hairs. Thereby, the hairs will be blacker and have more vitality.

The various objects and advantages of the present invention will be more readily understood from the following detailed description when read in conjunction with the appended drawings, in which:

### BRIEF DESCRIPTION OF THE DRAWINGS

FIG. 1 is a perspective view of the present invention;

FIG. 2 is an exploded perspective view of the present invention;

FIG. 3 is a cross-sectional view of the present invention;

FIG. 4 is a perspective view according to another embodiment of the present invention; and

FIG. 5 is a cross-sectional view according to another embodiment of the present invention.

### DETAILED DESCRIPTION OF THE PREFERRED EMBODIMENTS

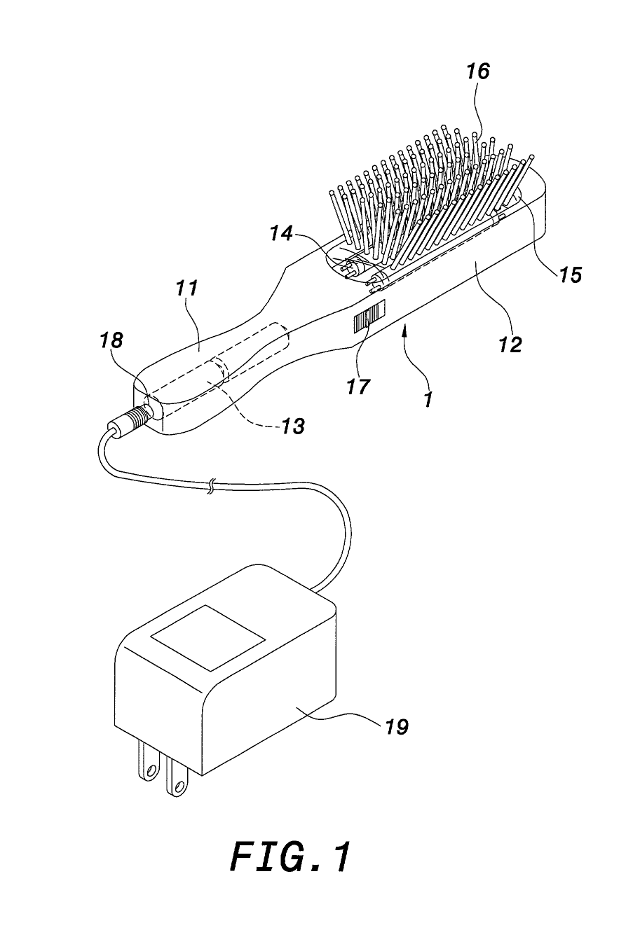

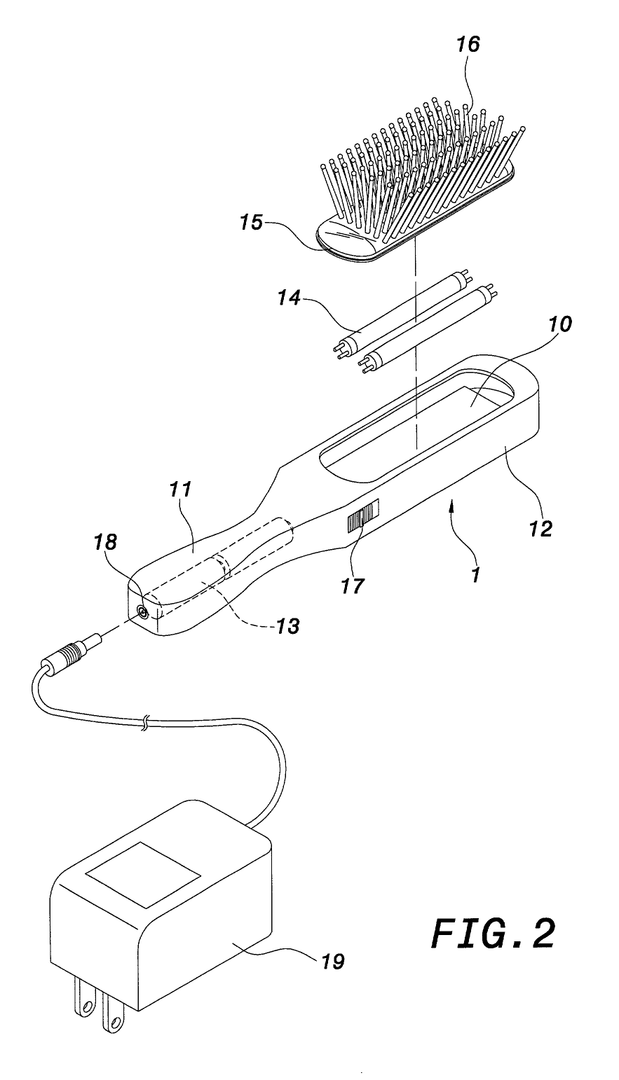

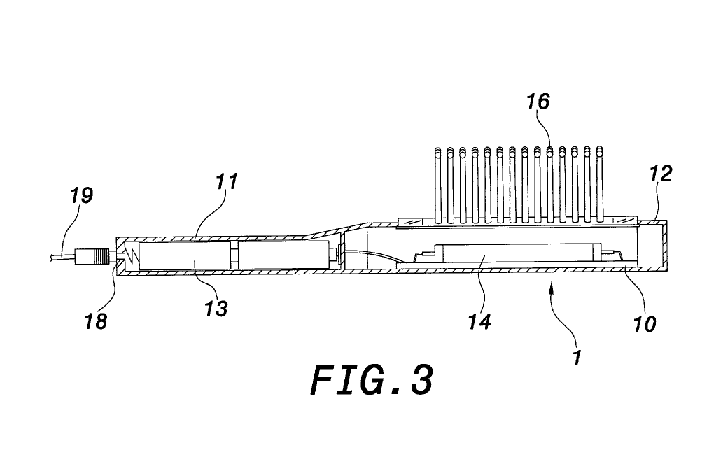

FIGS. 1 to 3 show a device having a far IR radiator for enhancing the growth of hair according to a first embodiment of the present invention, which device comprises a main body 1 having a hold part 11 and a contact part 12. A hollow room is formed in the hold part to receive a battery 13. A hollow room is also formed in the contact part to receive a far IR radiator 14. The far IR radiator 14 can be installed on a circuit board 10. A transparent cover 15 can be further arranged on the outer surface of the contact part 12. A comb 16 having arrays of comb fingers is joined on the cover 15. The main body 1 has a switch 17 and a power socket 18. The power socket 18 is connected to an AC power source 19. Electricity is provided by the battery 13 or an outer power source connected to the power socket 18 to let the far IR radiator 14 emit far IR lights for stimulating the skin of the scalp. Therefore, the hairs can be protected and nourished, and hair follicles can be activated to grow new hairs so that the hairs will be blacker and denser and have more vitality.

The far IR radiator 14 can be a lamp tube, i.e., a lamp tube with material capable of emitting far IR lights coated on the outer surface or the inner surface thereof. When the lamp tube is turned on, far IR lights can be emitted. If the main body is a closed and opaque body, a transparent cover 15 must be installed so that far IR lights can be transmitted out. Additionally, the far IR radiator 14 can be a ceramic plate. When the ceramic plate is heated, far IR lights can be emitted. Apparently, the far IR radiators can also be other structures.

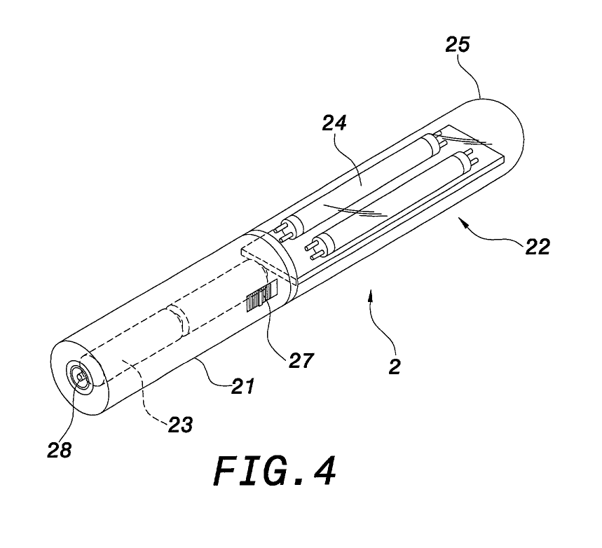

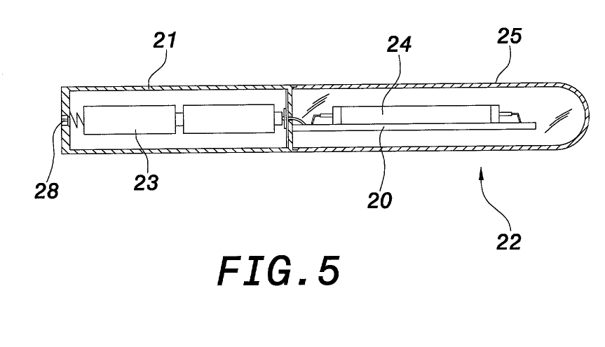

FIGS. 4 and 5 show a device having a far IR radiator for enhancing the growth of hair according to a second embodiment of the present invention, which device comprises a main body 2 having a hold part 21 and a contact part 22. A battery 23 is installed in the hold part 21. A far IR radiator 24 and a circuit board 20 are installed in the contact part 22. A transparent cover 25 is further arranged on the outer surface of the contact part 22. The main body 2 has a switch 27 and a power socket 28. The power socket 28 can be connected to a DC power source or an AC power source.

To sum up, the present invention exploits a far IR radiator capable of emitting radiation, which has slight heat and also far IR lights. Far IR lights having the effect of activating cells can be provided to penetrate into 1.5 centimeters below the skin. This depth is much larger than that required for the application of the present invention. The scalp of the human body is a thin layer. Because the scalp must suffer the brain pressure, the problem of insufficient supply of nutriments usually occurs, resulting in bad quality of the hairs. If the new comb structure of the present invention shown in FIGS. 1 to 3 is adopted, the scalp can be stimulated by heat and far IR lights when combing hairs so that hypodermic hair follicles can be activated. Once the vitality is increased, the human body will increase the supply of nutriments to the scalp so that the hair quality can be enhanced. Part gray hairs will thus become black ones, and part hair follicles will be activated to grow new hairs. Moreover, better nutriments can be supplied to common hair follicles so that the hairs will be denser, softer, and blacker. Of course, the simple irradiation type device shown in FIGS. 4 and 5 can also be adopted so that bald people can have better non-contacting way of use.

Although the present invention has been described with reference to the preferred embodiments thereof, it will be understood that the invention is not limited to the details thereof. Various substitutions and modifications have been suggested in the foregoing description, and others will occur to those of ordinary skill in the art. Therefore, all such substitutions and modifications are intended to be embraced within the scope of the invention as defined in the appended claims.

I claim:

1. A device having a far infrared radiator for enhancing the growth of hair, comprising:
a main body having a hold part and a contact part, a power source being installed in said hold part, a switch being installed on said main body; and
a far infrared radiator installed in said contact part, said power source letting said far infrared radiator emit far infrared lights to enhance the vitality of hypodermic hair follicles of the scalp.

2. The device having a far infrared radiator for enhancing the growth of hair as claimed in claim 1, wherein a battery is installed in said hold part of said main body.

3. The device having a far infrared radiator for enhancing the growth of hair as claimed in claim 1, wherein a transparent cover is further arranged on the outer surface of said contact part of said main body, and a comb having arrays of comb fingers is joined on said cover.

4. The device having a far infrared radiator for enhancing the growth of hair as claimed in claim 1, wherein a power socket is installed on the outer surface of said hold part of said main body.
